(19) United States
(12) Patent Application Publication
(10) Pub. No.: US 2011/0022126 A1
(43) Pub. Date: Jan. 27, 2011

(54) Trigeminal Nerve Stimulation Systems and Methods of Use

(76) Inventor: Stephen Taylor, Benson, AZ (US)

Correspondence Address: KAMMER BROWNING PLLC, 7700 BROADWAY, SUITE 202, SAN ANTONIO, TX, 78209, US

(21) Appl. No.: 12/843,038

(22) Filed: Jul. 25, 2010

Related U.S. Application Data
(60) Provisional application No. 61/228,555, filed on Jul. 25, 2009.

(51) Int. Cl. A61N 1/36

(52) U.S. Cl. 607/61

(57) ABSTRACT

The present invention discloses systems and methods for stimulation of the trigeminal nerve via indirect application of micro-current from a self-contained frequency/static-charged device. The device is treated with a process that allows it to store and dispense a low-level charge. The device can be directly or indirectly infused with a frequency from an external charging system; embedded with a charge-sustaining supplement and infused with a frequency that can be transferred from an external charging system, or embedded with a frequency generating and sustaining component, without infusion from an external charging system. The device operates independently once infused or embedded with operational frequency. The device is positioned near one or more trigeminal access point such as intra-oral, temple, or neck. The device may be used to increase strength, flexibility, balance, calm heart rate, and help control pain. Usage encourages neural plasticity, establishing sustainable new connections between nerve cells.

### CROSS REFERENCE TO RELATED APPLICATIONS

This application claims the benefit under Title 35 United States Code §119(e) of U.S. Provisional Application No. 61/228,555 filed Jul. 25, 2009, the full disclosure of which is incorporated herein by reference.

### BACKGROUND OF THE INVENTION

1. Field of the Invention

The present invention relates generally to devices for providing increased muscle strength, comfort, and relaxation through the use of micro-current on various body parts. The present invention relates more specifically to a device for the indirect application of micro-current to the trigeminal nerve via a self-contained electrostatically charged appliance with no ongoing external electrical energy source required. Such devices may be intra-oral or proximal to the skin of the temple or neck.

2. Description of the Related Art

A number of efforts have been made in the past to utilize an energy source in the treatment of pain, tension, and various medical and psychiatric conditions. Many of these methodologies involve the use of radio frequency electromagnetic energy, resonant acoustic energy, or therapeutic resonant frequencies. Such systems are used to treat disorders of the immune system, musculoskeletal system, cancer, malnutrition, and depression, to name only a few. Such devices include everything from simple mouth guards to more complex oral appliances and electromagnetic generators.

There are, of course, multiple examples in the prior art of mouth guards and other oral appliances. Many of these are for protection of the teeth and gums during athletic activities. Others are designed to improve performance through increased muscle strength and endurance or to correct or improve a disability or disorder such as temporomandibular joint (TMJ) syndrome. Other prior art devices are designed to combine the use of an energy source with an oral appliance but are not directed to the trigeminal nerve. All of these devices utilize an electrical energy source (continuous or intermittent) as part of the therapy.

Electromagnetic fields are commonly used to treat or suppress various medical disorders and maladies, and are typically applied through electrodes which are directly in contact internally or externally to a target area of a patient's body. Prior art has recognized that electronically stimulating specific points in the mouth is beneficial in treating sleep disorders, stress, TMJ problems, bruxism (U.S. Pat. No. 6,954,668), obstructive sleep apnea (U.S. Pat. No. 6,618,627), snoring (U.S. Pat. Re. 36,120), and Bell's Palsy (U.S. Patent Application Publication US 2008/0082131).

Previous implementations in the prior art characteristically include a power source, an oscillator (or other frequency-generating device), electrodes, a control device, an optional timer, and a host appliance that position the electrodes in the target area. In cases where an alternating current is uses as the stimulus for an oral device, the frequency used in the prior art is generally between 5 and 1,200 Hz.

Another passive device in prior art has recognized the trigeminal nerve as a thermal contact area that can be used to treat headaches (U.S. Pat. No. 5,693,077). Also recognized is the method of removing pressure, or de-stimulating, the trigeminal nerve to reduce or minimize the effects of Tourette's Syndrome, TMJ, displaced articulated disks, headaches, sleep disorder breathing, and vertigo.

In general, the prior art does not address the need for a performance enhancing device that calms the heart rate, provides increased relaxation and comfort, is non-invasive, readily usable by the general population (e.g. doesn't require configuration or customization to suit a particular user), low cost, self-contained, and which doesn't require ancillary or supplemental devices (e.g. power supplies, frequency generators, etc.) that immobilize or hinder the user's movement while using the apparatus.

### SUMMARY OF THE INVENTION

The present invention provides systems and methods for stimulation of the trigeminal nerve with a self-contained frequency/static-charged apparatus. The apparatus fits in various host appliances and does not require ancillary or supplemental devices. The self-contained frequency transfer device is used to increase a person's overall strength, increase flexibility, improve balance, calm the heart rate and to some extent help control pain.

The device consists of a contact pad or other apparatus that has been treated with a process that allows the device to store and dispense a low-level static charge used to stimulate the trigeminal nerve. The trigeminal nerve is stimulated by the device making contact with the oral cavity or the skin of specific areas of the temple or neck. The device is self contained, in that once it has been treated with the process, no external connections or appliances are necessary for the device to work. Additionally, the device works with or without any additions or modifications.

The infusion process can be applied to any device or apparatus that fits into the mouth or which makes contact with the trigeminal nerve access points on the exterior of the body, such as the temples and neck. The device stimulates the trigeminal nerve via a stored electronic frequency. Apparatuses such as mouth guards, dentures, dental retainers, teeth whitening strips, and any other products that fit into the mouth for the protection and/or care of the jaw, gums and teeth may be used. These include oral devices in the prior art that increase performance, improve flexibility, reduce pain, prevent bruxism, or improve balance, as these devices can also be used in the infusion process. The external devices include, but are not limited to helmets, hats, headbands, necklaces, eye glasses, goggles, or any other appliance, apparel, or item that makes contact with the trigeminal nerve access points.

The device can be infused with a frequency/static charge in a number of ways, including direct contact with an external charging system, exposure to an electrostatic energy field, embedded with a frequency generating and sustaining component, or embedded with a charge-sustaining component and charged by an external charging system.

In all of these embodiments, the device operates independently once infused or embedded with the operational charge. Once a device is charged, it can be worn or applied in such a manner that the treated material is in proximity or in contact with one or more of the trigeminal nerve access points. Use of the device enhances neural plasticity, establishing new connections between nerve cells which are sustained after use of the device.

### BRIEF DESCRIPTION OF THE DRAWINGS

The device, method of frequency infusion, and detailed description of operation are best understood with reference to the drawing figures. For the purpose of illustration and explanation, general embodiments of the device are shown. It should be understood that these illustrations do not limit the exact form and application of the device, the specific instrumentation and method used to infuse the device with a frequency, nor the benefits or advantages not specifically identified but realized during use of the device.

FIG. 1 depicts a typical mouth guard, which is one intra-oral embodiment charge storage device of the present invention.

FIG. 2 depicts a tooth whitening strip, which is one intra-oral embodiment charge storage device of the present invention.

FIG. 3 is depicts a dental retainer, which is one intra-oral embodiment charge storage device of the present invention.

FIG. 4 depicts a section of the human brain, identifying the stimulation path of the trigeminal nerve.

FIG. 5 is a block diagram of a first system of the present invention for carrying out a direct contact method of infusion of the device with a charge.

FIG. 6 is a block diagram of a second system of the present invention for carrying out a direct contact method of infusion of the device with a charge.

FIG. 7 is a schematic representation of the human head depicting the trigeminal nerve access points external to the human body.

FIG. 8 depicts a cap, which is one external embodiment charge storage device of the present invention.

FIG. 9 depicts eyeglasses, which is one external embodiment charge storage device of the present invention.

FIG. 10 depicts a neck tie, which is one external embodiment charge storage device of the present invention.

FIG. 11A is a block diagram of a third system of the present invention for carrying out an indirect method of infusion of the device with a charge.

FIG. 11B is a block diagram of a fourth system of the present invention for carrying out an indirect method of infusion of the device with a charge.

FIG. 12 is a flow chart of the methodology of the present invention.

### DETAILED DESCRIPTION OF THE PREFERRED EMBODIMENTS

The present invention provides systems and methods for stimulation of the trigeminal nerve with a self-contained frequency/static-charged apparatus. The apparatus is applied to the intra-oral cavity or specific areas of the temples or neck in order to stimulate one or more of the trigeminal nerve access points.

### Intra-Oral Devices

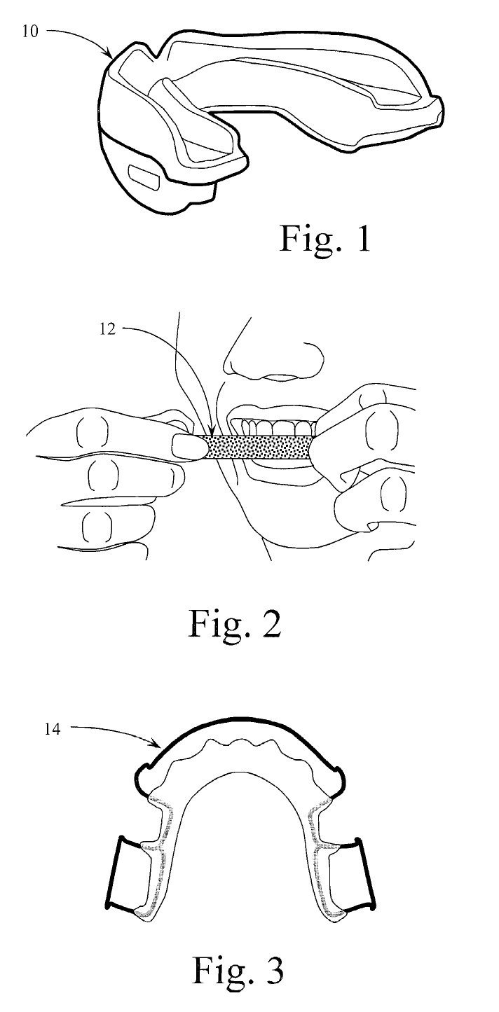

The intra-oral devices may include mouth guards 10, teeth whitening strips 12, dental retainers 14 (see FIGS. 1, 2, and 3) and any other products that fit into the mouth for the protection and/or care of the jaw, gums and teeth. The device can be used as a protective apparatus necessary to protect the jaw, mouth and teeth such as those required in contact sports. The device can be incorporated in night dental anti-bruxing guards and other sport and career apparatuses such as diving snorkels and muscular strengthening devices.

The frequency-induced device may be purchased as a mouth guard, dental whitening strip, or any other oral device commonly available to the public. The purchased device can be fitted by the consumer using common means (e.g. boiling, in the case of a mouth guard), or optionally by a qualified physician, dentist, or professional trainer (although not required). This makes the device mass-producible and readily available to all professional and amateur athletes, as well as patients who suffer from bruxing and sleep problems, at a greatly reduced price when compared to electrode-based frequency generation devices described in the prior art. Dental appliances and other professionally-fitted appliances can also be infused with the stimulating frequency using the methods described herein. This provides an option for a strength-potentiating device to be discreetly worn which can be used by people other than those participating in athletics.

Alternatively, the mouth guard may be molded from a composition including a pliable foundation material and a potentiated frequency-conducting material. The potentiate may be a contiguous piece or aggregated throughout the mouth guard and may be impregnated into or formed as an exterior composite.

The device may be infused into a pliable plastic material that is mixed with silica sand, gold, copper, diamond, ruby, quartz, sterling silver, or other conducting metals in the form of flakes, chips, or wires. An infusion of a compound having anti-bacterial mineral properties such as colloidal silver may also be put into the device (and other intra-oral frequency sustaining materials) to help reduce bacterial and viral growth.

### External Charge Storage Devices

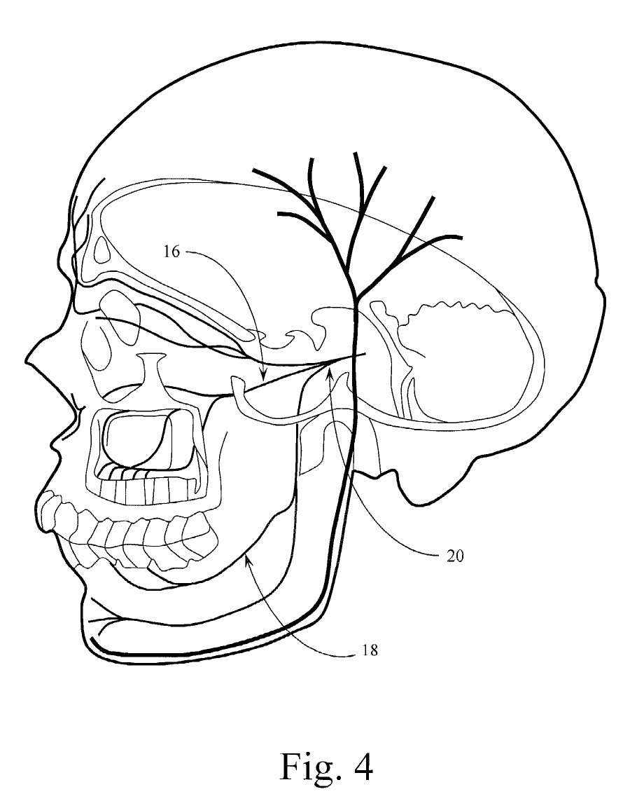

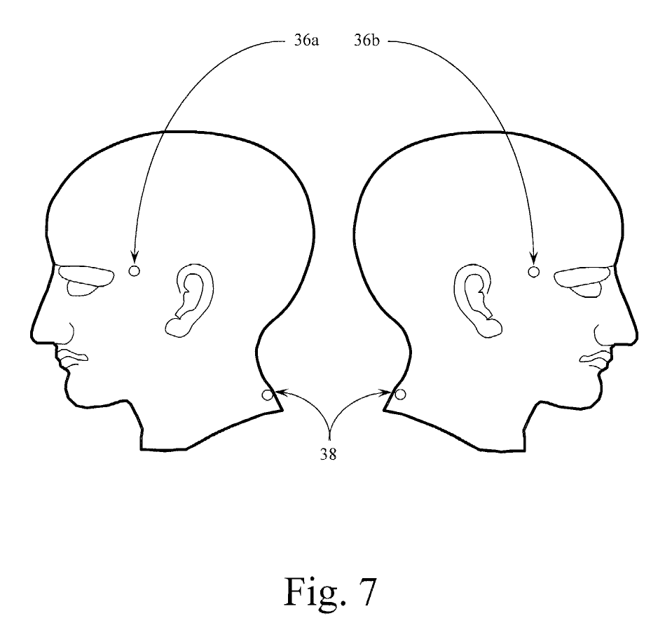

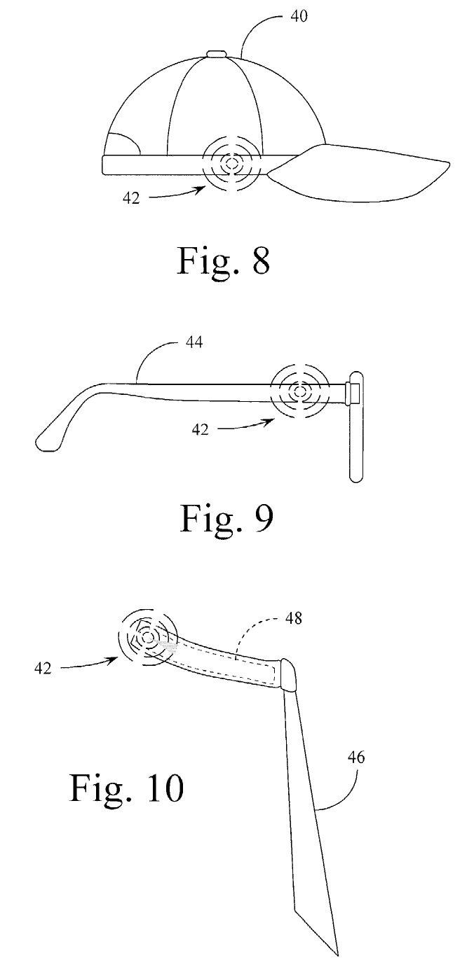

The external charge storage devices of the current invention have a self contained frequency/static charge and may be any of a variety of items which make contact with the trigeminal nerve access points on the exterior of the body. The stimulation pathways of the trigeminal nerve 20 are shown in FIG. 4 (see maxillary branch 16 and mandibular branch 18). FIG. 7 shows the trigeminal nerve access points external to the body, including the back of the neck 38, and the area over the temporal lobes 36a and 36b. The location of the trigeminal nerve access points facilitates the use of external charge storage devices that are commonly worn on the head, such as a cap 40 (see FIG. 8), the face, such as eyeglasses 44 (see FIG. 9) or neck, such as a neck tie 46 (FIG. 10). In each of these devices, the area of electromagnetic field effect 42 may be seen. It is this area which is positioned in proximity to one or more of the trigeminal nerve access points. In FIG. 10, the integrated conductive strip 48 is shown on the neck tie. Thus, any apparatus or apparel that makes contact with any of the trigeminal nerve access points can be used or augmented for the device, including helmets, hats, headbands, necklaces, eye glasses, or goggles.

Hats or protective headgear that touch the temporal lobes can be used a device, including military helmets, sports helmets, fire/hazard helmets, jacket hoods, head covers (such as “skull caps” or “do rags”), caps, and cycling helmets. These items would demonstrate the same area of electromagnetic field effect 42 which is shown in FIG. 8.

Face-wear is commonly secured to the face using arms that cross the temporal lobes and rest on the ears, or by a strap that stretches behind the head. Any apparatus that uses either of these methods of securing the face-wear to the face can be used as a device and would demonstrate the same area of electromagnetic field effect 42 as is shown in FIG. 9. A treated device, such as swimmer's goggles, will retain its effectiveness while dry or while submerged in water, therefore swimmer's goggles, diving masks, and any other type of goggles intended to be used in the water are applicable host apparatuses. Military applications include head and eye protective equipment, including goggles and helmets.

Eyeglasses and sunglasses 44 are applicable host apparatuses, including those constructed of metal, plastic, or composite material. Ski goggles can be augmented by attaching a treated conductive strip to the elastic strap of the goggles. The strips may contain adhesive backing, and may be in the form of a roll, pre-cut strips, or pre-cut shaped pieces. The trigeminal nerve access areas located on the temporal lobes can be energized through the types of devices worn on the head or as glasses or goggles as previously described.

The device can also be applied to another trigeminal nerve access area located in the back of the neck through any host apparatus that makes contact with this point. Possible devices include scarves, bowties, and neckties 46. Any shirt with a collar can be used to attach a treated conductive strip 48.

The device can be infused into a pliable plastic material that is mixed with silica sand, gold, copper, diamond, ruby, quartz, sterling silver, or other conducting metals in the form of flakes, chips and wires. The pliable plastic material can be molded into any shape that can be fitted or attached to the trigeminal nerve access areas.

While the device is to be used primarily to increase overall muscular strength, agility and stamina of the people using it, limited clinical research has shown that in some cases it has helped to control blood pressure, improve balance, reduce pain, reduce heart rate and improve sleep. The use of the device may improve balance, walking problems (gait), and motion sickness. Although there are potential benefits for all users of the device, it is not intended to replace the necessary diagnosis and treatment of chronic bruxism, temporal mandibular joint disorders, and sleep apnea. The device is also an effective supplement to neurostimulation, which is a medical treatment for people suffering from chronic pain associated with trigeminal neuralgia and other facial neuralgias.

### Description of the Device'S Neurological Effects and Benefits

The nervous system is directly influenced by neuro-sensory receptors. Once a receptor is brought to threshold (a frequency generated process), it fires the corresponding nerve. Mechanical receptors include equilibrium, hearing, arterial pressure, as well as all muscle contractions and relaxations. These receptors may take the form of free nerve endings found in the epidermis and dermis of the skin. Other types of receptors include Merkles Disc that have a large number of neuro-receptors innervating a single nerve fiber. Because of their large number of receptors all feeding the same nerve fiber, it takes far less stimulation to bring the nerve to threshold.

The tactile senses include touch, pressure, and vibration. All of the different tactile receptors are involved in detecting vibration. Pacinian corpuscles can signal vibrations from 30 to 800 cycles/sec., before they respond extremely rapidly to minute and rapid deformation of the tissues. Low frequency vibrations up to 80 cycles/sec. stimulate other tactile receptors, especially Meissner's corpuscles, which are less rapidly adapting than pacinian corpuscles.

The previous description illustrates that the frequency of a given vibration can be achieved through the skin and muscle fibers that affect the ophthalmic and maxillary branches of the trigeminal nerve as well through the mandibular branch of the mouth. Research has shown that the branches of the cranial nerve are also influenced by vibration stimulation. The majority of these branches are deeper in origin therefore are likely more influenced by the pacinian corpuscles.

The previous description also suggests that the frequency of the nerve fiber itself can be brought to threshold, without the need of receptor stimulus if the vibration is adequate enough to do so. This is explained by Arthur C Guyton, MD in his textbook “Basic Neuroscience”, edition. Dr. Guyton writes “Basically, any factor that causes sodium ions to begin to diffuse inward through the membrane in sufficient numbers will set off the autonomic, regenerative opening of the sodium channels. This can result from simple mechanical disturbance of the membrane, chemical effects on the membrane, or passage of electricity through the membrane. All of these are used at different points in the body to elicit nerve or muscle action potentials: mechanical pressure to excite sensory nerve endings in the skin, chemical neurotransmitters to transmit signals from one neuron to the next in the brain, and electrical currant to transmit signals between muscle cells to include skeletal, cardiac and smooth muscle.”

The present invention discloses the use of a frequency/static charge that can be incorporated into a device that can raise the central integrative state of the neuron by affecting the and cranial nerves. By raising the central integrative state of the neuron, it requires a lower stimulus to bring the nerve to threshold and therefore lower stimulus to fire the neuron. Through this frequency stimulation, neuro-plasticity occurs and sets up a re-excitation process necessary for rhythmicity (see Guyton above). The device is able to raise the central integrative state by neuro-receptor potentiation and/or by directly affecting the permeability of the neuron itself, independent of the neuro-receptor. This stimulates the higher neuronal (brain) centers.

The human body's cellular function is dependent upon the frequencies of energy that it is producing or responding to. By incorporating specific electrical, light and sound frequencies into these devices, the use of the device increases the overall muscular strength and stamina of all of the muscles of the human body. This is accomplished by potentiating the maxillary and mandibular branches of the trigeminal nerve, therefore directly increasing potentiation of the reticular formation of the brain stem. It is this area of the brain that is responsible for the coordination of blood pressure, heart activity, vascular tone, expiration and inspiration function, pain control, initial digestive function, sleep and wakefulness. Potentiation is herein described as the persistent increase in synaptic strength following high-frequency and/or static discharge stimulation. The devices disclosed in the present invention have the ability to retain long term potentiation in combination with the addition of the conducting material.

The current device applies a low-amperage frequency in the range of 2,000 Hz (2 KHz) to 15,000 Hz (15 KHz) to the intra-oral cavity or areas of the head and neck, thus stimulating the trigeminal nerve. These frequencies and static charge increase the physiological functions stated above and increase the myo-fascicular function of all the skeletal muscles. This usually increases the overall strength of the user by 20% to 25%. More specifically, the device stimulates a group of mixed neuroreceptors including mechanical, thermal, touch, stretch and visceral. These neuroreceptors then potentiate the afferent neuro-pathways of the trigeminal nerve affecting the motor, sensory and proprioceptive centers of the pons, brainstem and mid-brain region.

A significant distinction of the current device from the prior art is the sustaining neurological benefits that are realized by the user after the device has been worn and then removed. It is well established that nerve cells can form new connections with other nerve cells through a neurological principle called neural plasticity. Nerve connections are formed or modified by excitement of the nerve through mental, emotional, or physical stimulation. The stimulus triggers the nerve cells into creating the most efficient pathway to the brain and the nervous system to produce a response to the given stimulus. Once the stimulus is removed, the newly formed or modified nerve connections remain intact, improving the efficiency of the neural pathways.

It is this ability of adaptation of the nervous system that makes the device work. By placing any embodiment of the device near the trigeminal access points, neural plasticity occurs which increases the presynaptic excitation of the terminal of the neuron or neurons directly stimulated by the device. These presynaptic terminals contain many vesicles and mitochondria. Mitochondria produce energy for the nerve cells through cellular respiration, and are rich in fats, proteins and enzymes. The distance between the presynaptic and postsynaptic terminal is known as the synaptic cleft, which is part of the neuronal synapse. Synaptic vesicles secrete “neurotransmitters” into the synaptic cleft. This is the site of functional apposition between neurons, which is an impulse transmitter from one neuron to another by an electrical or chemical means.

In a typical synapse, the impulse is transmitted by a neurotransmitter (acetylcholine, norepinephrine, etc.) released by the excited presynaptic cell which diffuses across the synaptic cleft to bind with receptors on the postsynaptic cell membrane and thereby effects electrical changes in the postsynaptic cell which results in depolarization (excitation) or hyperpolarization (inhibition). The presynaptic terminal secrets a neurotransmitter and this transmitter acts upon receptor proteins in the membrane of the postsynaptic terminal to excite or inhibit the neuron. Synapses always transmit their impulses in one direction from the presynaptic terminal to the postsynaptic terminal.

A nerve impulse is an electrical event. Transference of the event is a product of time with important information transferred faster that non-important information. Conduction and speed of conduction is dependent upon the size of the individual nerve fiber and whether it is myelinated or non-myelinated. Myelin is a lipid (fat and sugar composition) and protein sheath surrounding the larger nerve fibers. The myelinated nerves allow for faster nerve transmission.

The nerve cell membrane is semi-permeable with a higher concentration in its resting state of sodium on the outside (extracellular) and potassium on the inside (intracellular). Conduction between the intra and extra cellular fluids follows the pathway of least resistance. A potential is defined as power which is present and ready for action, but not yet active. When bodies of different potentials are brought into communication, a current is set up between them. If they have the same potential, no current passes between them.

An action potential is the electrical activity developed in a nerve cell during activity. It may be elicited by electrical, chemical or mechanical stimulation, by temperature change, by change in oxygen availability, and so on. The membrane potential is the electrical potential that exists on the two sides of the membrane or across the wall of the cell. The spike potential is the initial, very large change in potential of an excitable cell membrane during excitation. As there is increased sodium on the outside of the cell, sodium, being a cation, carries a positive charge. As sodium migrates to the inside of the membrane, it brings a positive charge to the inside. As potassium, which is also a cation, is displaced by the inflow of sodium from outside to the inside, potassium then migrates to the outside of the membrane.

Most electrical properties occur at the cellular membrane. Nerves act as if they were composed of parallel capacitors. Capacitors consist of two conductors separated by an insulator. The insulator is the membrane with the lipids being sandwiched between the layers of protein. The membrane acting as a capacitor can store and discharge charges. A nerve cell can be depolarized or hyperpolarized. In the depolarization of a nerve membrane, the resistance to sodium influx becomes less and the inside of the nerve fiber becomes more positive. In hyper polarization, the resistance to sodium inflow increases and the inside of the nerve becomes more negative. The resting potential is when the cell is not being stimulated. In human beings, there is no true resting potential as the nervous system is continually active.

Nerve signals are transmitted by action potentials, which are rapid changes in the membrane potential. Each action potential begins a sudden change from the normal resting negative potential to a positive membrane potential and then ends with an almost equally rapid change back to the negative potential. This process is extremely fast, happening over a few 10,000ths of a second.

The successive stages of the action potential are resting, depolarization, and repolarization. The resting stage is the resting membrane potential before the action potential occurs. The membrane is said to be “polarized” during this stage because of the very large negative membrane potential that is present. The depolarization stage occurs when the membrane suddenly becomes very permeable to sodium ions, allowing tremendous numbers of sodium ions to flow to the interior of the axon, the normal “polarized” state of −90 mV is lost, with the potential rising rapidly in the positive direction. In large nerve fibers, the membrane potential actually overshoots beyond the zero level and becomes somewhat positive, but in smaller nerve fibers as well as many central nervous system neurons, the potential merely approaches the zero level and does not overshoot to the positive state. The repolarization stage occurs within a few 10,000ths of a second after the membrane becomes highly permeable to sodium ions, the sodium channels begin to close, and the potassium channels open more than normally. Then, rapid diffusion of potassium ions to the exterior re-establishes the normal negative resting membrane potential. This is called repolarization of the membrane.

The device of the present invention actuates the depolarization and repolarization stages by stimulating the trigeminal nerve with a frequency that equates to a millivoltage charge. The device, due to the pre-set infusion of a micro-current charge, sets up an electrical frequency directly affecting and bringing to threshold mechanical receptors, primarily the merkles disc and meissners corpuscles. It is these receptors that are activated by touch, taste, and slight pressure, changing the membrane potential of the receptor, and bringing it to threshold.

The charge potentiates the superior alveolar branches (anterior, middle, posterior) of the infraorbital nerve, and the inferior dental plexus (inferior dental and gingival nerves). The lingual nerve also receives stimulation. Receptors initiate neuro-conduction only when an adequate stimulus is present to which the receptor has the lowest threshold. As the device provides the required stimulus, then the receptor, which is graded, initiates an action potential to the nerve fiber.

In essence, the device is much like an alternator or generator in a car, in that it completes and recharges the necessary function of the car's engine to run more efficiently. The device actually performs differently from individual to individual because it is regulated by the body's nervous system and there are no two people, hence nervous systems, exactly alike.

### Description of the Device'S Frequency Infusion Process

The device is infused with a self-sustaining frequency and charge using any of several infusion systems and methods. One method for infusing the apparatus comprises direct or indirect coupling of a frequency charge using a frequency generation source and electrodes. The device can be infused with a frequency transferred from an external charging system, resulting in the device being autonomously operational after the initial frequency transfer from the charging system.

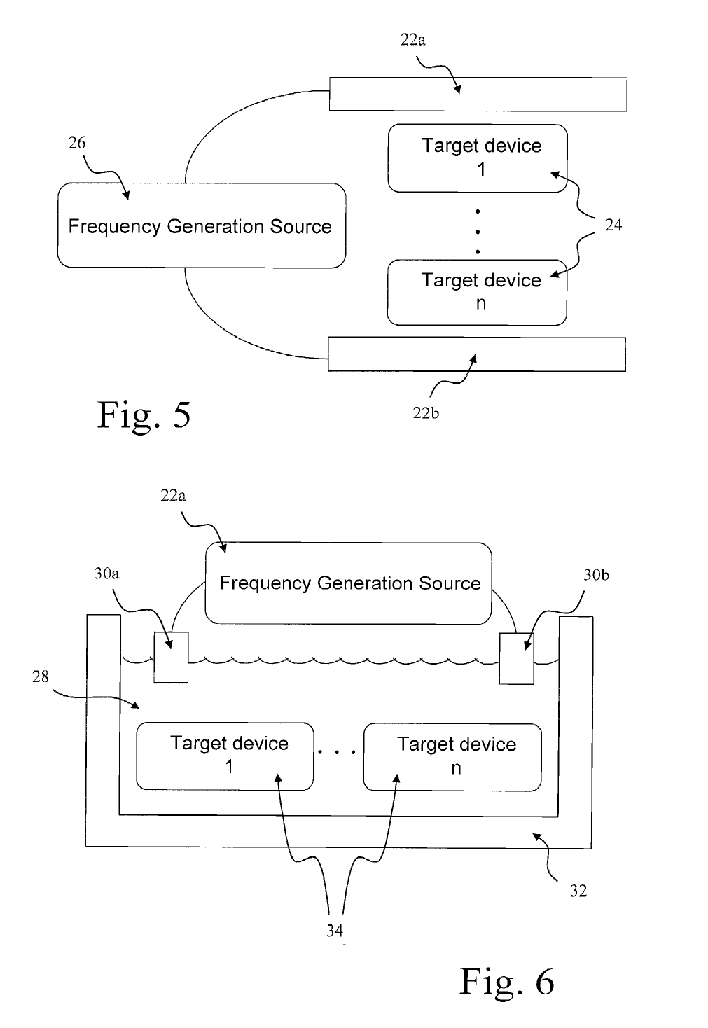

In one embodiment of the infusion system, one or more target apparatuses are placed in direct contact (see FIG. 5) with electrodes 22a and 22b that transfer a frequency and charge to the target device(s) 24 from a frequency generation source 26. The source can be any equipment component that generates an output frequency (e.g. oscillator, frequency generator, including wind machine, computer, or any embodiment of a frequency generating device), preferably, but not required, at a selectable rate (frequency) and voltage level. After a prescribed time, the target apparatus retains the transferred charge from the source, and the device can be used in the manner previously described. In another embodiment of the infusion system (see FIG. 6), one or more target devices 34 are placed in a container 32 filled with a water bath 28, and water-compatible electrodes or adaptors 30a and 30b are placed in the water bath to transfer a frequency and charge to the target device(s) from a frequency generation source 22a. In other embodiments of the present invention, common dental and/or medical equipment that produce, use, or transfer frequency in their application (such as ultrasound, x-ray, or other imaging equipment) can also be used.

In alternate embodiments, the apparatus is injected with a supplemental compound, microchip, crystal, silica sand, oscillator, or other frequency generating or sustaining component, and subsequently infused with a frequency or charge, wherein the apparatus is capable of retaining a frequency charge. For example, the device may be implanted with silica sand, conductive metal, or other composition to increase intensity and/or extend the length of time the device will hold the frequency charge. In another embodiment, the device is embedded with a microchip, oscillator, crystal, or another frequency generating and sustaining component, without the need to be infused from an external charging system.

Alternatively, the device may be infused with a frequency/static charge through exposure to an electrostatic energy field. The energy field transfers a low-voltage residual static charge to the device. The treated device retains the static charge indefinitely depending on the properties of the device material.

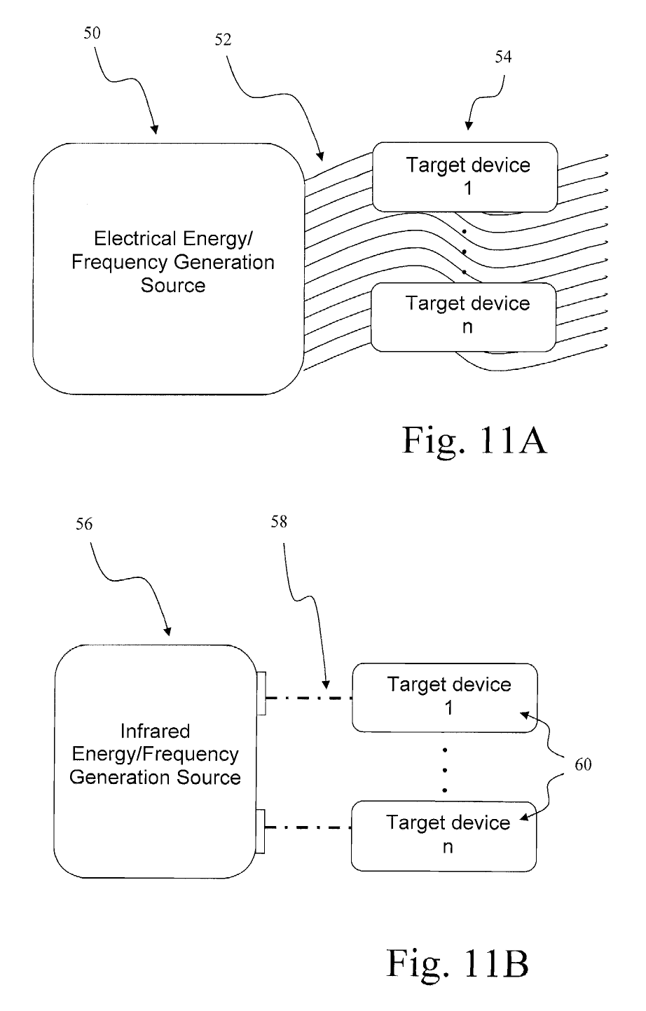

In another embodiment of the infusion system, one or more target host apparatuses are placed in the effective radiating area of an electrical (see FIG. 11A) or infrared (see FIG. 11B) energy/frequency generation source (preferably having a selectable frequency and power level). The source, radiating area, and target devices may be housed in a self-contained enclosure, or may also be an open area with the source being a self-contained unit. In FIG. 11A, the electrical energy 52 of the electrical energy frequency generation source 50 is sufficient to transfer a frequency and static charge to the target device(s) 54 within the effective radiating area. In FIG. 11B, the infrared energy 58 of the infrared energy frequency generation source 56 is sufficient to transfer a frequency and static charge to the target device(s) 60 within the effective radiating area. After a prescribed time, the target apparatus retains the transferred charge from the source, and the device can be used in the manner previously described. A further embodiment of the infusion system is through the use of a spray.

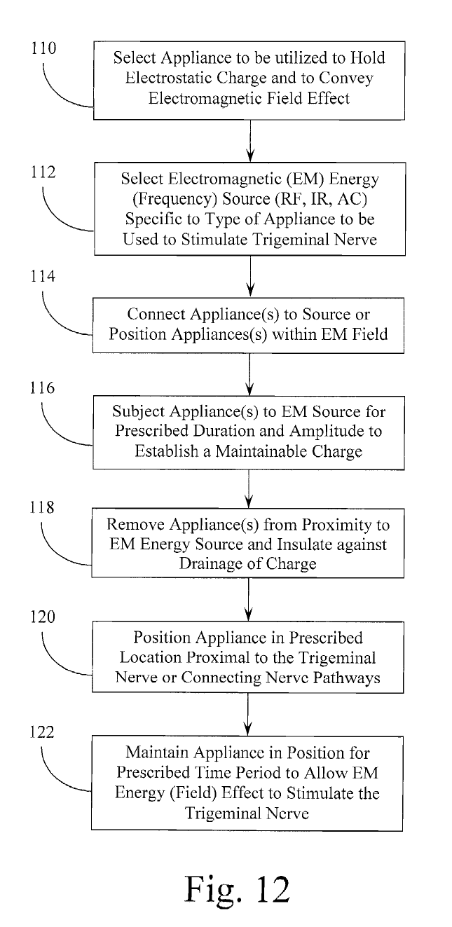

As shown in FIG. 12, the methodology of the present invention for improving the function of neurological pathways and improving physical performance in a human user by stimulation of the trigeminal nerve and/or branches thereof, includes the steps of: (a) selecting at least one energy storage appliance to be utilized to hold an electrostatic charge and convey an electromagnetic (EM) field effect to stimulate the trigeminal nerve 110; (b) selecting a fluctuating EM energy source specific to a type of energy storage appliance selected to be used to stimulate the trigeminal nerve 112; (c) positioning the energy storage appliance in close proximity to the selected fluctuating EM energy source 114; (d) subjecting the selected energy storage appliance to an influx of energy from the selected fluctuating EM energy source for a prescribed duration at a prescribed amplitude and frequency to establish a maintainable energy charge within the selected energy storage appliance 116; (e)removing the energy storage appliance from close proximity to the EM energy source and insulating against drainage of the established energy charge 118; (f) positioning the selected energy storage appliance in a prescribed location on the human user proximal to the trigeminal nerve and/or branches thereof 120; and (g) maintaining the selected energy storage appliance in position for a prescribed time period to allow the EM field effect to stimulate the trigeminal nerve and/or branches thereof 122.

Although the present invention has been described in terms of the foregoing preferred embodiments, this description has been provided by way of explanation only and it is not intended to be a limitation of the invention. Those skilled in the art will recognize modifications of the present invention that might accommodate specific requirements of the user and, as such, do not necessarily depart from the spirit and scope of the invention. The present invention may be embodied or implemented in different forms without departing from the nature and intent of the device described herein. The illustrations should not be construed as conclusive description of form, function, or scope of the device.

I claim:

1. A system for improving the function of neurological pathways and improving physical performance in a human user, the system comprising:
an energy storage device configured to be positioned in close proximity to the trigeminal nerve and/or branches thereof in the human user; and
a means for infusing an energy charge into the energy storage device, the energy charge sufficient to stimulate an electromagnetic field effect in the trigeminal nerve and/or branches thereof when the energy storage device is positioned in close proximity thereto.

2. The system of claim 1 wherein the energy storage device comprises a device configured for intra-oral placement within the human user.

3. The system of claim 2 wherein the intra-oral device comprises a sports mouth guard made of a resilient polymer material configured to be held between the upper and lower teeth of the human user.

4. The system of claim 2 wherein the intra-oral device comprises an adhesive backed strip for placement over the surfaces of the teeth of the human user.

5. The system of claim 2 wherein the intra-oral device comprises a professionally fitted dental appliance configured to be placed and held in conjunction with the teeth and/or gums of the human user.

6. The system of claim 1 wherein the energy storage device comprises a device configured for external placement on the head and/or neck of the human user.

7. The system of claim 6 wherein the external device comprises a component of, or the entirety of, an article of apparel or an accessory thereof that is worn over, and at least partially covers, the head of the human user.

8. The system of claim 6 wherein the external device comprises a component of, or the entirety of, an article of apparel or an accessory thereof that is worn over, and at least partially covers, the face of the human user.

9. The system of claim 6 wherein the external device comprises a component of, or the entirety of, an article of apparel or an accessory thereof that is worn around, and at least partially covers, the back of the neck of the human user.

10. The system of claim 1 wherein the means for infusing an energy charge into the energy storage device comprises a direct contact AC voltage source having a frequency.

11. The system of claim 1 wherein the means for infusing an energy charge into the energy storage device comprises a non-contact fluctuating electromagnetic field generator having a frequency.

12. The system of claim 10 wherein the means for infusing an energy charge into the energy storage device further comprises electrically conductive electrodes for placement in direct contact with the one or more energy storage devices arranged in parallel or series.

13. The system of claim 10 wherein the means for infusing an energy charge into the energy storage device further comprises electrically conductive electrodes for placement in a liquid bath within which the one or more energy storage devices are immersed.

14. The system of claim 11 wherein the means for infusing an energy charge into the energy storage device comprises a radio frequency (RF) oscillator generating a fluctuating electromagnetic (EM) field within the space about the one or more energy storage devices.

15. The system of claim 11 wherein the means for infusing an energy charge into the energy storage device comprises a infrared (IR) light source generating light waves in the infrared spectral frequency range into the space about the one or more energy storage devices.

16. A method for improving the function of neurological pathways and improving physical performance in a human user by stimulation of the trigeminal nerve and/or branches thereof, the method comprising the steps of:
(a) selecting at least one energy storage appliance to be utilized to hold an electrostatic charge and to thereby convey an electromagnetic (EM) field effect to stimulate the trigeminal nerve and/or branches thereof;
(b) selecting a fluctuating EM energy source specific to a type of energy storage appliance selected to be used to stimulate the trigeminal nerve and/or branches thereof;
(c) positioning the at least one selected energy storage appliance in close proximity to the selected fluctuating EM energy source;
(d) subjecting the at least one selected energy storage appliance to an influx of energy from the selected fluctuating EM energy source for a prescribed duration at a prescribed amplitude and frequency to establish a maintainable energy charge within the at least one selected energy storage appliance;
(e) removing the at least one selected energy storage appliance from close proximity to the EM energy source and insulating against drainage of the established energy charge;
(f) positioning the at least one selected energy storage appliance in a prescribed location on the human user proximal to the trigeminal nerve and/or branches thereof; and
(g) maintaining the at least one selected energy storage appliance in position for a prescribed time period to allow the EM field effect to stimulate the trigeminal nerve and/or branches thereof.

17. The method of claim 16 wherein the step of selecting at least one energy storage appliance to be utilized to hold an electrostatic charge and to thereby convey an electromagnetic (EM) field effect to stimulate the trigeminal nerve and/or branches thereof, comprises infusing a commercially available object with electrostatic energy retention compounds.

18. The method of claim 16 wherein the fluctuating EM energy source comprises an AC voltage source having a frequency and the step of positioning the at least one selected energy storage appliance comprises establishing an electrically conductive path through the at least one selected energy storage appliance with electrodes.

19. The method of claim 16 wherein the fluctuating EM energy source comprises an EM wave generator having a frequency and the step of positioning the at least one selected energy storage appliance comprises placing the same within the fluctuating EM field generated by the EM energy source.

20. The method of claim 16 wherein the step of positioning the at least one selected energy storage appliance in a prescribed location on the human user comprises placing the at least one appliance in an intra-oral position.

21. The method of claim 16 wherein the step of positioning the at least one selected energy storage appliance in a prescribed location on the human user comprises placing the at least one appliance in an external position adjacent the back of the neck and/or one or both of the temporal lobes.
